(19) United States
(12) Patent Application Publication
(10) Pub. No.: US 2009/0053694 A1
(43) Pub. Date: Feb. 26, 2009

(54) Photochemically Amplified Bioassay

(76) Inventor: Leo B. Kriksunov, Ithaca, NY (US)

Correspondence Address: PASTEL LAW FIRM;CHRISTOPHER R. PASTEL, 8 PERRY LANE, ITHACA, NY, 14850-9267, US

(21) Appl. No.: 11/306,378

(22) Filed: Dec. 26, 2005

(51) Int. Cl. C12Q 1/68; G01N 33/53; G01N 33/555

(52) U.S. Cl. 435/6; 435/7.1; 436/520

(57) ABSTRACT

In the present invention, a reagent capable of immunospecific reaction with the analyte of interest is conjugated to a photocatalytic microparticle. After the immunospecific binding has occurred, the assay amplification is performed by exposing photocatalytic particles to actinic UV light in presence of an oxidizable compound. Photocatalytic particles are catalyzing multiple occurrences of oxidation of oxidizable compound under UV light irradiation resulting in detectable changes such as color change. This provides for amplification of each single act of immunospecific binding and is followed by colormetric detection. Thus a high sensitivity quantitative or qualitative immunoassay can be realized.

### FIELD OF INVENTION

The present invention relates to assays for an analyte, such as an antigen or an antibody, in a liquid sample, such as body fluid. More particularly, the present invention relates to a method and device for amplified detection of an analyte in a liquid sample using an amplification technique utilizing a photo-catalytic compound or particle, which catalyses a reaction which results in amplified colorimetric detection of the analyte.

### BACKGROUND OF INVENTION

Many types of ligand-receptor assays have been used to detect presence of various substances in body fluids such as urine or blood or pathogens in the environment. Such assays typically involve antigen-antibody reactions and conjugates comprising fluorescent, chemiluminescent, radioactive, magnetic, enzymatic, or colored, visually observable tags or labels on immunoreactive species, which serve as an indicator that an immunospecific reaction has occurred. In most of these assays, there is a receptor (e.g. an antibody) which is specific for the selected antigen (or an antigen receptor for the selected antibody), and means for detecting the presence and/or amount of the antigen-antibody reaction product.

The detection tags are often conjugated to the receptor, or less frequently, carried within sacs such as animal erythrocytes, polymer microcapsules, or liposomes. In the lateral flow immunoassays, optical or visual tags are extensively employed, such as colored metal micro- or nanoparticles, for example gold particles, which create a visible spot or a line when immunoreaction has occurred. This visual tag is observable by naked eye, but usually serves only as a qualitative indicator of the presence of the analyte. More recently, paramagnetic metal particles were introduced which can be detected by magnetic means. Also recently, optically detectable quantum dots were proposed as indicators of the presence of the analyte also suitable for multiplexing of assays.

Immunoassays can be divided into two broad categories of non-amplified assays and amplified assays. Non-amplified assays involve the use of a tag, such as fluorescent tag, chemical specie tag, electrochemical tag, radioactive label, metal particle, or the like on immunoreactive species which serves as a direct indicator that an immunospecific reaction has occurred. Only one tag per occurrence of immunospecific binding is activated, precipitated, or released for subsequent detection by optical, chemical, electrochemical, magnetic, etc. means or by detection of radiation. One of the main disadvantages of the non-amplified assays is low sensitivity of assays, false positives and false negatives, and generally ambiguous results in case of low concentrations of the analyte. As a consequence, larger sample volumes and concentrating of the sample may be required for detection of extremely low concentrations of analytes.

This problem is addressed by amplified assays which involve amplification of each binding act between analyte and the immunoreagent. For example, enzyme-linked immunosorbant assays (ELISAs) involve the use of an enzyme covalently coupled to an immunoreactive reagent to serve as an indicator that an immunospecific reaction has occurred. The enzyme is capable of catalyzing a chemical reaction, resulting in a detectable chemical change. Importantly, the enzyme is capable of many (hundreds) catalyzed reaction or turnover events in a reasonable period of time. The high sensitivity of ELISA is due to the number of turnover events the enzyme is capable of during an incubation period with a substrate that is reacting to result in a colored or easily detectable reaction product. While ELISA type assay can be extremely sensitive, it is frequently a very time-consuming assay sensitive to storage and processing conditions, which is difficult to use in the field. ELISA may also require significant laboratory skill to perform the assay.

Another type of an amplified assay is electrochemiluminescence (ECL) based assay, where an electrochemical tag is covalently coupled to an immunoreactive reagent and reacts electrochemically to emit light signal to serve as an indicator that an immunospecific reaction has occurred. When stimulated by an applied electrical current on an electrode, the electrochemical tag undergoes many reversible redox reactions/turnover events, emitting light signal each time, thus amplifying each single occurrence the immunospecific reaction.

Yet another type of an amplified assay is assay based on method of immunoanalysis which combines immobilized immunochemistry with the technique of flow injection analysis, and employs microscopic spherical structures such as microcapsules or liposomes (lipid vesicles) as carriers of detectable reagents. For example, liposomes can be modified on their surface with analytical reagents, and carry in their internal volume a large number of fluorescent or electrochemically active tags. After the immunospecific reaction has occurred, the liposomes are lysed by contact with a liposome lysing agent such as surfactant solution or by another method, and release large amount of tags per each immunospecific binding act. The presence of tags is then detected by chemical, optical, electrochemical, or other means known in the art.

Some of the disadvantages of amplified assays described above are as follows. ELISA is a very time-consuming assay which is difficult to use in the field. The ECL requires complex and expensive equipment to read the assay, which is also difficult to use in the field. Amplified liposome-based assays are sensitive to storage conditions and require additional step of lysing the liposome to develop and read the assay. These techniques have not been able to solve all of the problems encountered in amplified and in rapid detection methods.

Applying a different classification, various methods for detecting the presence of an analyte in a sample of biological fluid through the use of immunochemistry can be classified as sandwich and competition assays. Assay broadly known as a “sandwich” method, are widely used whereby, for example, a target analyte such as an antigen, is “sandwiched” between a labeled antibody and an antibody immobilized onto a solid support. The assay is then read by observing the presence and amount of bound antigen-labeled antibody complex, which is typically detected by optical means. In the “competition” immunoassay method, antibody or another binding reagent is bound to a solid surface and then is contacted with a sample containing an unknown quantity of antigen analyte and with labeled antigen of the same type as analyte. The amount of labeled antigen bound on the solid surface can be then determined, usually by optical means, to provide an indirect measure of the amount of antigen analyte in the sample, as a difference between total binding ability of on the substrate and the amount of labeled antigen. These methods as well as other methods discussed below can typically detect both antigens and antibodies and these methods are generally known as immunochemical ligand-receptor assays or simply immunoassays.

Solid phase immunoassay devices, whether sandwich or competition type, provide sensitive detection of an analyte in a biological fluid sample such as blood or urine. Such devices typically incorporate a solid support to which one member of a ligand-receptor pair, usually an antibody, antigen, or hapten, is bound. Typical solid supports include microplate well walls, microbeads, as well as a number of porous materials such as nylon, nitrocellulose, cellulose acetate, glass fibers, and other porous polymers.

A number of self-contained immunoassay kits are using porous materials as solid phase carriers of immunochemical components such as antigens, haptens, or antibodies have been described. These kits are typically dipstick, flow-through, or migratory in design.

In a common form of dipstick assays, for example such as in some pregnancy detection kits, immunochemical components such as antibodies are bound to a solid phase. The assay device is “dipped” for incubation into a sample suspected of containing antigen analyte. Enzyme-labeled antibody is then added, usually after a brief incubation period. The device then is washed and immersed into a solution containing a substrate for the enzyme. The enzyme-label, if present, interacts with the substrate, causing the formation of colored products which are typically easily detectable by naked eye.

Flow-through type immunoassay devices decrease the need for extensive incubation and complicated washing steps usually associated with dipstick assays by incorporating these steps into the operation of assay device, with sample and reagents moving through the device driven by capillary forces. U.S. Pat. No. 4,632,901 discloses a device comprising a specific antibody bound to a porous membrane. As the liquid analyte samples added and flows through the membrane, target analyte binds to the antibody. The addition of sample is then followed by addition of labeled antibody. The visual detection of labeled antibody provides an indication of the presence of target antigen analyte in the sample. Such flow-through assays also known as migration type assays, or lateral flow assays, are typified by a membrane which is impregnated with the reagents needed to perform the assay. An analyte detection zone is provided in which analyte is bound and reacts with labeled (typically colored) reagent whereby assay results are read in the same detection zone. See, for example, U.S. Pat. No. 4,366,241, and U.S. Pat. No. 4,770,853. Typical labels include gold sol particles, U.S. Pat. No. 4,313,734, dye sol particles, U.S. Pat. No. 4,373,932, dyed latex particles, and dyes encapsulated in liposomes for amplification, such as described in U.S. Pat. No. 4,703,017.

### BRIEF DESCRIPTION OF THE INVENTION

Briefly, this invention provides an improved detection device and method having greater sensitivity and discrimination for analytes of interest as well as an assay device which is simple to manufacture. An amplified immunoassay is provided, which utilizes a catalyzing particle or molecule which can produce easily detectable color-based or optical detection-based amplification. The assay can be realized in a lateral flow or migration-type assay format, immobilized or test plate format, similar to ELISA or ELISPOT assay, a microfluidic device format, or another assay format.

In the present invention, a reagent capable of immunospecific reaction with the analyte of interest is conjugated to a photocatalytic microparticle, such as titanium dioxide microparticle. After immunospecific binding has occurred, assay amplification is performed by exposing photocatalytic particles to actinic UV light in presence of an oxidizable compound. Catalytic particles are catalyzing multiple occurrences of oxidation of oxidizable compound under UV light irradiation, resulting in detectable chemical or physical changes such as color change, pH change, conductivity change, or similar. This provides for amplification of each single act of immunospecific binding in an easy-to-read detection format, such as colorimetric detection. Thus a very high sensitivity quantitative or qualitative immunoassay can be realized.

### BRIEF DESCRIPTION OF DRAWINGS

FIG. 1 shows an embodiment of labeled antibody and labeled antibody-antigen complex according to present invention.

FIG. 2 shows an embodiment of immobilization of labeled antibody-antigen complex on capture site.

FIG. 3 shows an embodiment of assay amplification step according to present invention.

FIG. 4 schematically illustrates an embodiment of assay detection step according to present invention.

FIG. 5 schematically illustrates an embodiment of assay detection step according to present invention.

FIG. 6 shows an embodiment of amplified lateral flow assay.

FIG. 7 shows an embodiment of amplified assay realized in a microplate well.

### DETAILED DESCRIPTION OF THE INVENTION

Capture Step of Assay

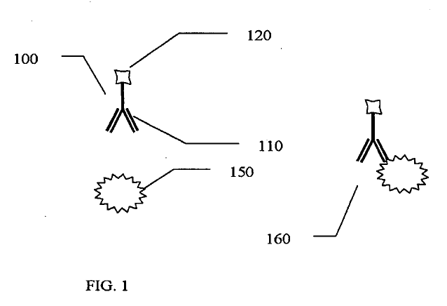

Referring now to FIG. 1, a “sandwich” assay embodiment of the present invention is illustrated. A reagent such as an antibody 110 is conjugated to a photocatalytic particle 120 to form a labeled antibody or reagent 100. The size of photocatalytic particles 120 is selected based on the ability to conjugate these particles to antibody 110 without significantly impeding the mobility of the resulting conjugate. The preferred size of photo-catalytic particles 120 is from several nanometers to hundreds of micrometers. Methods of conjugating particles to immunospecific reagents, such as antibodies or antigens, are well known to these skilled in the art. Another method to attach photocatalytic micro- or nanoparticles to antibody 110 is to encapsulate such particles in microcapsules, such as lipid vesicles or liposomes, and then conjugate liposomes to antibody 110 as it is known in the art. Yet another way of making labeled antibody 100 is by chemically conjugating a photocatalytic molecule to antibody 110.

Labeled reagent antibody 100 is capable of binding to a specific epitope (not shown) on analyte of interest 150 to form a labeled antibody-antigen complex shown with reference numeral 160.

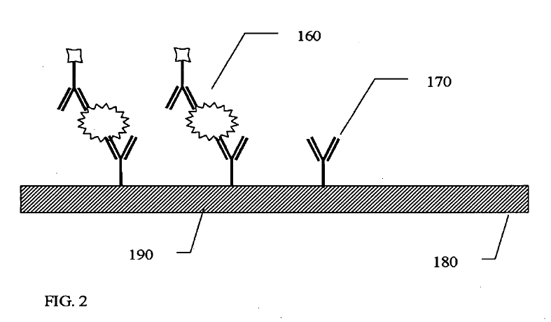

Referring now to FIG. 2, labeled antibody-antigen complex 160 is immobilized on a capture site 190 by reacting with an immobilized capture component or receptor 170 which, in this embodiment, is represented by a second antibody specific for the same or a different epitope of analyte 150. Capture component 170 is chemically bound or immobilized on solid phase support 180 as it is known in the art. Solid phase support 180 can be a surface of a microplate, surface of a micro-bead, or a wicking membrane. The methods for immobilizing immunospecific capture groups, such as antibodies or antigens, are well known in the art. Immobilizing of labeled antibody-antigen complex 160 on solid support 180 can be accomplished with reaction steps performed in a plurality of different sequences, including (a) first forming labeled antibody-antigen complex 160 and then reacting this complex with capture component 170; or (b) first capturing analyte antigens 150 by capture component 170 and then performing the reaction with labeled antibody 100; or (c) performing simultaneously the reactions between capture component 170, labeled antibody 100, and analyte 150.

The capture step of the assay is concluded once at capture site 190 on solid phase support 180, analyte 150 and labeled antibody 100 reacted with immobilized capture component 170 to form a “sandwich” thus immobilizing labeled antibody 100 through binding to analyte 150 which is in turn bound to capture component 170. The competitive assay technique wherein labeled analyte and any unlabeled analyte present in the sample compete for sites of attachment to solid support can be substituted for the sandwich assay technique within the framework of this invention, as it will be apparent to these skilled in the art.

In a lateral flow or migration assay embodiment of the present invention, labeled antibody 100—analyte 150—immobilized capture component 170 sandwich complex is progressively produced at capture site 190 as analyte 150 and labeled reagent 100, either already bound together to form labeled antibody-antigen complex 160 or independently, move through capture area 190 driven by capillary forces through a porous membrane. Similarly, in a microfluidic embodiment of present assay, analyte 150 and labeled reagent 100, either already bound together to form labeled antibody-antigen complex 160 or independently, move through capture area 190 driven by microfluidic pump, pressure gradients, electro-osmotic forces, or other means known in the art. As more and more labeled antibody 100 species are immobilized at capture site 190, photocatalytic particles 120 conjugated to antibody 110 accumulate on capture site 190. After all analyte 150 has reacted or passed capture site 190, the amplification step of the present assay is carried out.

In another embodiment of the present invention, present assay is realized in a well on a microplate. In this embodiment, the “sandwich” is forming in the well, immobilizing labeled antibody-antigen complex 160, and then all non-immobilized labeled antibodies 100 are removed from the microwell by a washing step. After this washing step, amplification step of present assay is carried out.

Amplification Step of the Assay

In the amplification step of the assay according to present invention, a catalytic reaction catalyzed by photocatalytic particles is initiated by irradiation with actinic UV light of capture area 190 where photocatalytic particles have been immobilized.

A number of photocatalytic compounds are known in the art, and a range of different photocatalytic particles or compounds can be employed in amplified assay according to present invention, including particles or photocatalytic compounds in molecular and not in corpuscular form. In a preferred embodiment of the present assay, Titanium Dioxide (TiO₂) microparticles are utilized. Titanium Dioxide is a known photocatalyst capable of catalyzing of various oxidation reactions such as oxidation of various organic compounds, typically in presence of water and/or oxygen. Several potential mechanisms of this catalytic oxidation were proposed, one of them being generation of highly oxidizing free radical species, such as hydroxyl radicals, from water and oxygen under influence of actinic UV light. Another proposed mechanism is a direct photocatalytic oxidation of organic compounds by air in presence of catalytic particles. Titanium dioxide has three polymorphs: rutile, brookite and anatase, of which anatase shows the highest photoactivity.

Titanium Dioxide is a semiconductor with the band gap energy of 3.2 eV. The photons having the energy >3.2 eV, corresponding to wavelengths <388 nm, which is UV light, (approximately 5% of the solar light reaching the surface of the earth has wavelengths <388 nm), can promote an electron from the valence to the conduction band which results in chemical reactions on the surface of TiO₂ creating either highly reactive hydroxyl radicals or superoxide ions, which both are very powerful oxidants capable to oxidize many organic and inorganic compounds.

In the amplification step of the present assay, under UV light irradiation, Titanium Dioxide photocatalytic particles produce strong oxidizing species in the immediate vicinity of photocatalytic particles. Longer exposure to UV light or higher intensity of UV light will generate more oxidizing species. Oxidizing species in turn are participating in oxidation reactions with oxidizable compounds present in the vicinity of photocatalytic particles, resulting in oxidation and changes in color due to oxidation or bleaching of colored organic compounds or dyes present or changes in color due to different coloration of oxidized vs. reduced form of a compound. For example, oxidation of iodide ion I⁻, present in the aqueous solution of potassium iodide, by oxidizing species, may produce iodine I₂. While iodide ion is colorless in aqueous solution, iodine has a strong coloration and can easily be detected and quantified by colorimetric means. Oxidation and bleaching of organic and inorganic compounds by UV light in presence of photocatalytic particles is known in the art. Other effects, including for example changes in solution pH due to photolysis of water in the vicinity of photocatalytic particles under UV irradiation, can also be detected and quantified by either pH measurements or corresponding coloration changes of a pH-sensitive pigment or pH-indicating pigment.

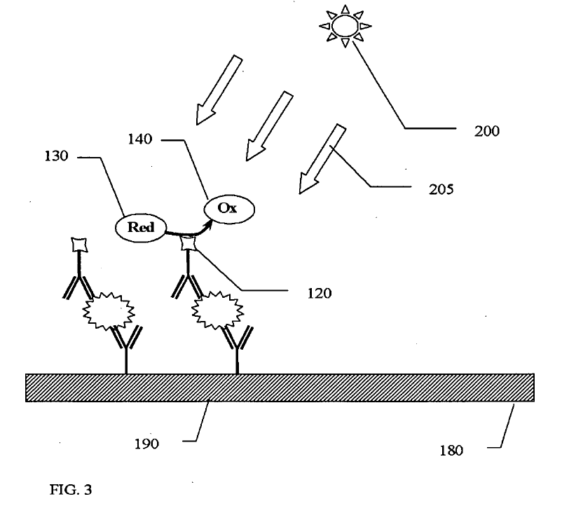

Referring now to FIG. 3, according to the present invention, UV light irradiation of immobilized photocatalytic particles 120 results in multiple catalyzed oxidation reactions in the vicinity of photocatalytic particles 120, which in turn result in detectable reaction products. In a preferred embodiment, oxidation reactions result in color change which is then used to read qualitative or quantitative assay results. As illustrated in FIG. 3, an actinic light from light source 200, containing at least some ultraviolet (UV) wavelength components, is irradiating capture area 190 with immobilized photocatalytic particles 120, with the direction of UV light schematically shown by arrows 205. The preferred light source 200 is a UV lamp, however any broad spectrum lamp, or even sunlight, can be utilized as a source of actinic light for the present assay.

An oxidizable compound 130 is present in the vicinity of photocatalytic particles 120. This compound is initially in its reduced (non-oxidized) state. One example of oxidizable compound 130 is a colored oxidizable organic dye which changes coloration upon oxidation or bleaching. In a lateral flow assay embodiment of the present invention, solid phase support 180 is a wicking membrane, and it has on its surface an immobilized organic dye or pigment as oxidizable compound 130.

Under influence of actinic light 200, oxidizable compound 130 is catalytically oxidized in the presence of photocatalytic particles 120 to form oxidized state 140 of the same compound. Importantly, the reaction of photocatalytic oxidation is taking place only when photocatalytic particles 120 are present, i.e. when analyte 150 of interest is present. In the absence of analyte 150 no photocatalytic particles 120 will be immobilized in capture area 190 and catalytic oxidation reactions cannot occur. Change (or absence of change) of coloration associated with the oxidation of oxidizable compound 130 such as organic dye is then detected and assay results are read in the detection step of the present assay. Oxidizable compound 130, such as organic dye can either be present in the solution in soluble form or can be immobilized on the surface of solid phase support 180.

In another embodiment of the present invention, oxidizable compound 130 is an inorganic compound which changes coloration upon oxidation, such as iodide ion as discussed above. In this embodiment, iodide ion is present in the buffer solution or is impregnated in the solid phase support 180, such as wicking membrane. Upon irradiation with UV light iodide ion is oxidized to form strongly colored iodine which is then detected and assay results are read in the detection step of the present assay. In yet another embodiment of the present assay, oxidizable compound 130 is a fluorescent compound capable of fluorescence. Upon oxidation or oxidative degradation of oxidizable compound 130, it loses fluorescent properties, which can then be detected and measured by methods known to these skilled in the art. Other oxidizable compounds, whose properties detectably change upon oxidation, can be employed in the present amplified assay.

Detection Step of the Assay

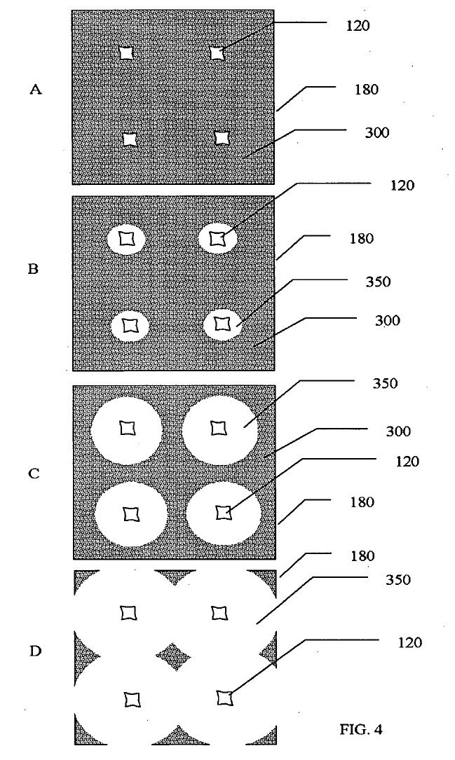

(currently amended) Referring now to FIG. 4, a top view of the assay in a sandwich format illustrates the effects and color changes upon irradiation with UV light. In FIG. 4A four photocatalytic particles 120 are schematically shown immobilized on solid phase support 180 [[190]] prior to UV light illumination. Solid phase support is shown colored by oxidizable organic dye 300. FIGS. 4B, 4C, and 4D schematically illustrate effects of exposure to UV light, with FIG. 4B illustrating initial exposure, FIG. 4C illustrating additional exposure, and FIG. 4D illustrating longest exposure to UV light in presence of photocatalytic particles 120. As it is illustrated in FIG. 4, bleached areas 350 will form around photocatalytic particles 120, with the size and discoloration of bleached areas 350 progressively increasing due to continuous catalytic oxidation of organic dye 300. Initially, only dye around particles 120 is oxidized and somewhat bleached, but with increased amount of time and/or intensity of actinic light 200, bleached areas 350 will increasingly discolor and increase in size.

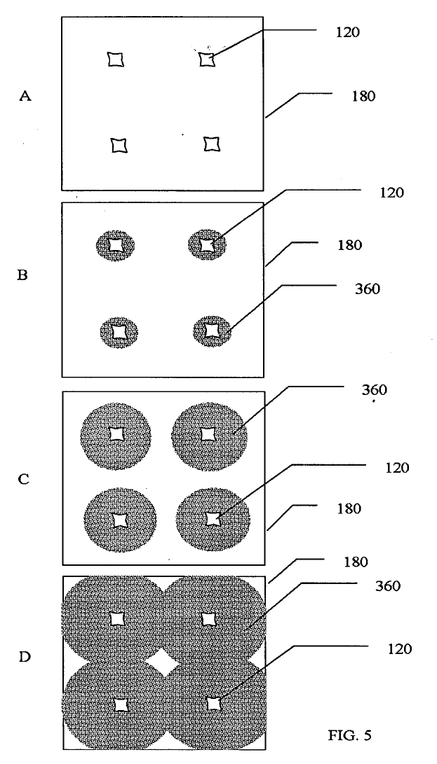

(currently amended) Referring now to FIG. 5, an embodiment of the present invention is shown. Oxidizable non-colored compound, such as iodide ion, is present in capture zone 190 and is becoming colored upon catalytic oxidation. A top view of the assay illustrates the effects and color changes upon irradiation with UV light. In FIG. 5A four photocatalytic particles 120 are schematically shown immobilized on solid phase support 180 [[190]] prior to UV light illumination. FIGS. 5B, 5C, and 5D schematically illustrate effects of exposure to UV light, FIG. 5B illustrating initial exposure, FIG. 5C illustrating additional exposure, and FIG. 5D illustrating longest exposure to UV light in presence of photocatalytic particles 120. As it is illustrated in FIG. 5, colored areas 360 consisting of oxidized form of oxidizable compound will grow around photocatalytic particles 120, with the size and depth of coloration of areas 360 progressively increasing due to continuous catalytic oxidation of oxidizable compound.

The change in coloration is then used for measuring the presence and/or quantity of analyte 150. Each immunobinding event, which results in one captured and immobilized photocatalytic particle 120, results in multiple reactions of catalytic oxidation, thus amplifying the reading of the assay. Detection of much smaller quantities of analyte 150, compared with non-amplified assays, is thereby possible.

As it would be apparent to these skilled in the art, the change in coloration can be detected and quantified by a variety of available optical techniques, for example visually, by plate readers, optical scanners, optical colorimeters, and other similar devices.

The amount of actinic light utilized to amplify and quantitatively measure analyte in the present assay can additionally be adjusted depending upon the quantity of the analyte present in the biological fluid sample. When very small amount of analyte is present, the intensity of actinic light and or exposure time can be increased resulting in stronger amplification of the assay. To the contrary, if relatively larger amount of analyte is present, the intensity of actinic light and/or exposure time can be decreased resulting in lesser amplification of the assay. Advantageously, the present invention enables additional amplification of the assay if higher sensitivity is required. After reading the assay, additional UV irradiation can be utilized to produce higher degree of amplification.

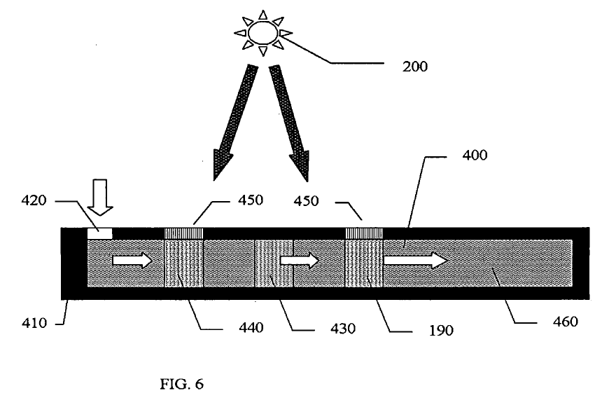

An embodiment of the present invention, whereas the immunoassay is realized in lateral flow format is further illustrated with reference to FIG. 6. A bibulous or non-bibulous wicking membrane 400 is encapsulated by non-wicking, liquid-impermeable device body 410. A port 420 is provided for the introduction of biological fluid sample, which is tested for potential presence of analyte 150. The movement of liquid sample through the device is illustrated by non-filled arrows. In this embodiment of the present invention, membrane 400 is colored by immobilized organic dye, which is applied either throughout the whole membrane or only in capture area 190 and calibration/test area 440. Area 430 contains unbound photocatalytic particle labeled antibody or reagent 100 capable of immunospecific binding to analyte. A biological fluid sample, moving through the device, encounters the area 430 where analyte 150, if present in the sample, immunospecifically reacts and binds to photocatalytic particle-labeled antibody 100 forming mobile labeled antibody-antigen complex 160. Biological fluid sample then continues to move towards wicking pad 460, passing through capture area 190. Labeled antibody-antigen complex 160 is then captured and immobilized in capture area 190 by receptors 170.

Area 440 optionally contains permanently immobilized photocatalytic particles 120 and can be utilized for calibration of the amplification of the present assay. Transparent windows 450 in device body 410 are utilized to UV-irradiate capture area 190 and calibration/test area 440 and to read the assay. After introduction of biological fluid sample and capture of labeled antibody-antigen complex 160, the device is subjected to UV irradiation form UV source 200 for a time sufficient to develop necessary amplification of the assay. Changes in coloration of capture area 190 and if necessary calibration area 440 are then read to determine presence and/or quantity of analyte 150.

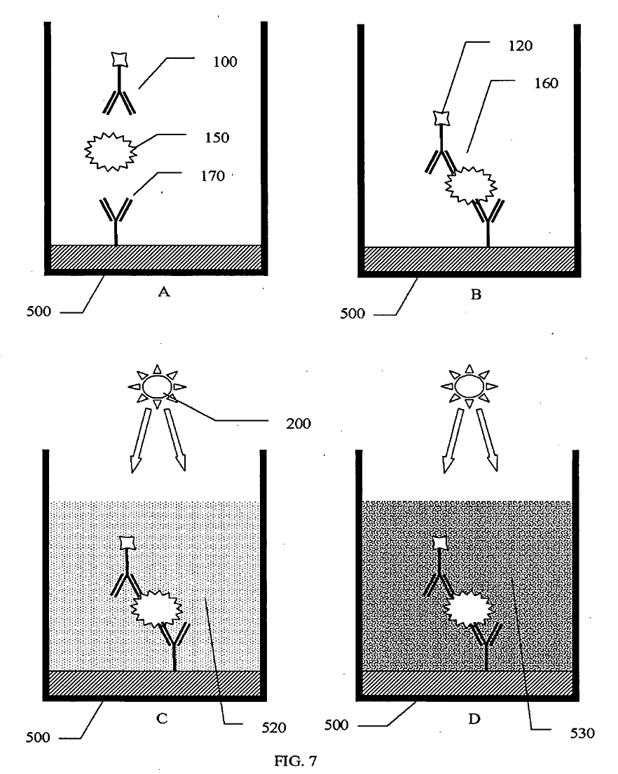

Referring now to FIG. 7, another embodiment of the present invention is illustrated where the assay is performed in a microplate well 500. FIG. 7A schematically shows microplate well 500 which contains labeled antibody or reagent 100, fluid sample with unknown quantity of analyte 150, and immobilized capture component or receptor 170. After reaction of immunospecific binding between reagent 100, analyte 150, and immobilized receptor 170 has occurred, labeled reagent 100 and photocatalytic particles 120 are immobilized in microplate well 500 as illustrated in FIG. 7B. This is followed by a washing step (not shown), whereby all unbound species are removed from microplate well 500.

Referring now to FIG. 7C, following washing step, a buffer solution 520 containing oxidizable compound is introduced into microplate well 500, and microplate well 500 is subject to actinic UV irradiation from UV light source 200. When photocatalytic particles 120 are present photocatalytic oxidation reaction of oxidizable compound is initiated. As further illustrated in FIG. 7D, continuing photocatalytic oxidation reaction will result in color changes due to conversion of oxidizable compound to oxidized form. Buffer solution containing oxidized form of oxidizable compound is shown with reference numeral 530 in FIG. 7D. The assay is then read by optical means by detecting color changes in microplate well as known in the art.

The assay of the present invention was illustrated by referring to an assay for one particular entity, e.g. an antigen. The general principles and techniques described here for assaying an antigen can be applied to assay for other species such as, for instance, antibodies, enzymes, proteins, haptens, DNA, RNA, etc. For example, these skilled in the art would appreciate that the same method would apply to detection of antibodies, with corresponding changes, whereby for instance instead of labeled antibody reagent a labeled antigen reagent can be used for detecting antibodies in analyte. Similarly, in this case instead of immobilized capture component being an antibody, capture component being an antigen can be employed. Additionally, catalytic reaction produced by photo-catalytic particle may not necessarily be a reaction resulting in color change, but any physical and/or chemical change, as discussed above.

From the foregoing description, one skilled in the art can easily ascertain the essential characteristics of this invention and, without departing from the spirit and scope of this invention, can make various changes and modifications of the invention to adapt to various usages and conditions. The present invention has been described in detail, including the preferred embodiments. However, it will be appreciated that those skilled in the art, upon consideration of the present disclosure, may make modifications and improvements on this invention and still be within the scope and spirit of this invention as set forth in the following claims.

1. A method for performing an assay of a fluid sample for potential presence of an analyte, which comprises the steps of:
(a) providing a solid phase support with immobilized receptors for binding said analyte,
(b) providing binding members labeled with photocatalytic particles, said binding members labeled with said photocatalytic particles capable of binding said analyte,
(c) providing an oxidizable compound capable of photocatalytic oxidation under irradiation by an ultraviolet light in the presence of said photocatalytic particles,
(d) permitting said analyte, said immobilized receptors, and said binding members labeled with said photocatalytic particles to react, forming immobilized photocatalytic particles,
(e) irradiating said immobilized photocatalytic particles with said ultraviolet light, photochemically oxidizing said oxidizable compound,
(f) detecting oxidation of said oxidizable compound, and
(g) repeating steps (e) and (f) at least once to increase amplification of said assay and to adjust amplification of said assay to concentration of the analyte.

2. The method according to claim 1, additionally comprising the step of removing said binding members labeled with said photocatalytic particles which have not reacted to form said immobilized photocatalytic particles,
wherein intensity of said ultraviolet light and time of irradiating of said photocatalytic particles are changed to adjust amplification of said assay to concentration of the analyte.

3. The method according to claim 1,
wherein said assay is a competitive assay or a sandwich assay,
wherein said assay is lateral flow assay,
wherein said oxidizable compound is bound to at least part of said solid phase support,
wherein permanently bound photocatalytic particles are provided on a part of said solid phase support and used for calibrating and controlling of amplification of said assay, and
wherein said assay is incorporated into a device body having at least one transparent window, said transparent window adapted to irradiate said immobilized photocatalytic particles and said permanently bound photocatalytic particles with said ultraviolet light.

4. The method according to claim 1,
wherein said assay is an immunoassay, a migration assay, a lateral flow assay, a microfluidic assay, or an assay in a microplate well format,
wherein irradiation by said ultraviolet light results in a change in pH, and
wherein said change in pH is detected by a pH-sensitive pigment.

5. The method according to claim 1, wherein said photocatalytic particles are metal, metal oxide, polymer, composite of metal and polymer, composite of metal oxide and polymer, photocatalytic molecule, or composite of photocatalytic molecule and polymer.

6. The method according to claim 1, wherein said photocatalytic particles comprise titanium dioxide.

7. The method according to claim 1, wherein said step of detecting oxidation of said oxidizable compound is performed by a detection means,
wherein said detection means is an optical reader, a microplate reader, a colormeter, or a naked eye.

8. The method according to claim 1, wherein said analyte is selected from a group consisting of an antigen, an antibody, a hapten, a nucleic acid, a protein, and an enzyme; and
wherein said immobilized receptors are selected from a group consisting of an antigen, an antibody, a hapten, a nucleic acid, a protein, and an enzyme, and
wherein said oxidizable compound is a fluorescent compound having fluorescent properties, and wherein upon oxidation said fluorescent compound loses said fluorescent properties, which is then detected in step (f) of said assay.

9. The method according to claim 1, wherein said oxidizable compound is a chemical compound that changes color when oxidized, and
wherein said oxidizable compound is an iodide ion forming iodine upon oxidation.

10. The method according to claim 1, wherein said oxidizable compound is a dye, a colorant, a water soluble dye, a fluorescent compound, an organic compound, or an inorganic compound.

11. A method for performing an assay of a fluid sample for potential presence of an analyte, which comprises the steps of:
(a) providing binding members labeled with photocatalytic particles, said binding members labeled with said photocatalytic particles capable of binding said analyte,
(b) providing an oxidizable compound capable of photocatalytic oxidation under irradiation by an ultraviolet light in presence of said photocatalytic particles,
(c) permitting said analyte to react with said binding members labeled with photocatalytic particles, forming analyte bound with said binding members labeled with photocatalytic particles,
(d) irradiating said analyte bound with said binding members labeled with said photocatalytic particles with said ultraviolet light, photochemically oxidizing said oxidizable compound, and
(e) detecting oxidation of said oxidizable compound.

12. The method according to claim 11, additionally comprising the step of removing said binding members labeled with said photocatalytic particles which have not reacted to form said immobilized photocatalytic particles.

13. The method according to claim 11, wherein said assay is a competitive assay or a sandwich assay.

14. The method according to claim 11, wherein said assay is an immunoassay, a migration assay, a lateral flow assay, a microfluidic assay, or an assay in a microplate well format.

15. The method according to claim 11, wherein said photocatalytic particles are metal, metal oxide, polymer, composite of metal and polymer, composite of metal oxide and polymer, photocatalytic molecule, or composite of photocatalytic molecule and polymer.

16. The method according to claim 11, wherein said photocatalytic particles comprise titanium dioxide.

17. The method according to claim 11, wherein said step of detecting oxidation of said oxidizable compound is performed by a detection means,
wherein said detection means is an optical reader, a microplate reader, a colormeter, or a naked eye.

18. The method according to claim 11, wherein said analyte is selected from a group consisting of an antigen, an antibody, a hapten, a nucleic acid, a protein, and an enzyme;
wherein said immobilized receptors are selected from a group consisting of an antigen, an antibody, a hapten, a nucleic acid, a protein, and an enzyme.

19. The method according to claim 11, wherein said oxidizable compound is a chemical compound changing color when oxidized.

20. The method according to claim 11, wherein said oxidizable compound is a dye, a colorant, a water soluble dye, a fluorescent compound, an organic compound, or an inorganic compound.
